(19) United States
(12) Patent Application Publication
(10) Pub. No.: US 2019/0029854 A1
(43) Pub. Date: Jan. 31, 2019

(54) HANDLE WITH DIRECT DRIVE MECHANISM

(71) Applicant: BOSTON SCIENTIFIC SCIMED, INC., MAPLE GROVE, MN (US)

(72) Inventors: Michael P. Calomeni, San Jose, CA (US); Randy S. Gamarra, Santa Clara, CA (US); Takashi H. Ino, San Jose, CA (US)

(73) Assignee: BOSTON SCIENTIFIC SCIMED, INC., MAPLE GROVE, MN (US)

(21) Appl. No.: 16/048,703

(22) Filed: Jul. 30, 2018

Related U.S. Application Data
(60) Provisional application No. 62/538,280, filed on Jul. 28, 2017.

(51) Int. Cl. A61F 2/962

(52) U.S. Cl. CPC A61F 2/962; A61F 2002/9517

(57) ABSTRACT

A handle for a medical device system may include a hollow elongate handle housing, a first lead screw disposed within the hollow elongate handle housing, a deployment mechanism configured to actuate a medical implant positioned at a distal end of the medical device system, the deployment mechanism being operatively engaged with the first lead screw, and a control knob rotatably disposed around a proximal end of the hollow elongate handle housing. The first lead screw may be spaced apart from the control knob. Rotation of the control knob in a first direction may cause rotation of the first lead screw in the first direction.

### CROSS-REFERENCE TO RELATED APPLICATIONS

This application claims the benefit of priority under 35 U.S.C. § 119 to U.S. Provisional Application Serial No. 62/538,280, filed Jul. 28, 2017, the entirety of which is incorporated herein by reference.

### TECHNICAL FIELD

The present disclosure pertains to medical devices, and methods for manufacturing and/or using medical devices. More particularly, the present disclosure pertains to configurations of a device for delivering a replacement heart valve.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first aspect, a handle for a medical device system may comprise a hollow elongate handle housing; a first lead screw disposed within the hollow elongate handle housing; a deployment mechanism configured to actuate a medical implant positioned at a distal end of the medical device system, the deployment mechanism being operatively engaged with the first lead screw; and a control knob rotatably disposed around a proximal end of the hollow elongate handle housing. The first lead screw may be spaced apart from the control knob. Rotation of the control knob in a first direction causes rotation of the first lead screw in the first direction.

In addition or alternatively, and in a second aspect, one full rotation of the control knob produces more than one full rotation of the first lead screw.

In addition or alternatively, and in a third aspect, the control knob includes a ring gear disposed along an inner surface of the control knob.

In addition or alternatively, and in a fourth aspect, the ring gear is fixedly attached to the control knob.

In addition or alternatively, and in a fifth aspect, the first lead screw includes a pinion gear at a proximal end of the first lead screw.

In addition or alternatively, and in a sixth aspect, the pinion gear is directly engaged with the ring gear.

In addition or alternatively, and in a seventh aspect, the pinion gear is fixedly attached to the proximal end of the first lead screw.

In addition or alternatively, and in an eighth aspect, rotation of the control knob in a second direction opposite the first direction causes rotation of the first lead screw in the second direction.

In addition or alternatively, and in a ninth aspect, the handle may further comprise a tubular collar disposed about a proximal portion of the hollow elongate handle housing, the tubular collar being rotatable relative to the hollow elongate handle housing and the control knob.

In addition or alternatively, and in a tenth aspect, a medical device system may comprise an elongate outer sheath; an elongate inner sheath disposed within a lumen of the outer sheath; and a handle disposed at a proximal end of the outer sheath. The handle may comprise a hollow elongate handle housing; a first lead screw disposed within the hollow elongate handle housing; a deployment mechanism configured to actuate a medical implant positioned at a distal end of the inner sheath, the deployment mechanism being operatively engaged with the first lead screw; and a control knob rotatably disposed around a proximal end of the hollow elongate handle housing. The first lead screw may be spaced apart from the control knob. Rotation of the control knob in a first direction causes rotation of the first lead screw in the first direction. Initial rotation of the first lead screw in the first direction causes proximal translation of the outer sheath relative to the inner sheath without actuating the medical implant.

In addition or alternatively, and in an eleventh aspect, subsequent rotation of the first lead screw in the first direction causes the deployment mechanism to actuate the medical implant without proximal translation of the outer sheath relative to the inner sheath.

In addition or alternatively, and in a twelfth aspect, the medical implant is a replacement heart valve implant.

In addition or alternatively, and in a thirteenth aspect, actuation of the replacement heart valve implant shifts the replacement heart valve implant from an elongated delivery configuration to an expanded deployed configuration.

In addition or alternatively, and in a fourteenth aspect, the medical implant is releasably secured to the deployment mechanism by at least one release pin.

In addition or alternatively, and in a fifteenth aspect, proximal translation of the at least one release pin releases the medical implant from the medical device system.

In addition or alternatively, and in a sixteenth aspect, a handle for a medical device system may comprise a hollow elongate handle housing; a first lead screw disposed within the hollow elongate handle housing, the first lead screw including a plurality of external teeth disposed at a proximal end of the first lead screw; a deployment mechanism configured to actuate a medical implant positioned at a distal end of the medical device system, the deployment mechanism being operatively engaged with the first lead screw; and a control knob rotatably disposed around a proximal end of the hollow elongate handle housing, the control knob including a plurality of internal teeth proximate a proximal portion of the control knob. The plurality of external teeth is directly engaged with the plurality of internal teeth. Rotation of the control knob in a first direction causes rotation of the first lead screw in the first direction.

In addition or alternatively, and in a seventeenth aspect, the plurality of external teeth is integrally formed with the first lead screw.

In addition or alternatively, and in an eighteenth aspect, the plurality of internal teeth is integrally formed with the control knob.

In addition or alternatively, and in a nineteenth aspect, the first drive screw is rotatable independently of the hollow elongate handle housing.

In addition or alternatively, and in a twentieth aspect, rotation of the control knob at a first rate produces rotation of the first lead screw at a second rate higher than the first rate.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example medical device system;

FIG. 2 illustrates selected components of an example medical implant associated with the medical device system;

FIG. 3 illustrates selected components of an example medical implant associated with the medical device system;

FIG. 4 illustrates an example handle associated with the medical device system;

FIG. 5 is a partially exploded view of the handle of FIG. 4;

FIG. 6 illustrates selected components of the example handle;

FIGS. 7-9 illustrate an example of coordinated movement of selected components within the example handle; and

FIG. 10 is a partial cut-away view illustrating selected components associated with the example handle.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve or the mitral valve can have a serious effect on a human and could lead to serious health condition and/or death if not dealt with properly. Treatment of defective heart valves poses other challenges in that the treatment often requires the repair or outright replacement of the defective valve. Such therapies may be highly invasive to the patient. Disclosed herein are medical devices that may be used for delivering a medical device to a portion of the cardiovascular system in order to diagnose, treat, and/or repair the system. At least some of the medical devices disclosed herein may be used to deliver and implant a replacement heart valve (e.g., a replacement aortic valve, replacement mitral valve, etc.). In addition, the devices disclosed herein may deliver the replacement heart valve percutaneously and, thus, may be much less invasive to the patient. The devices disclosed herein may also provide a number of additional desirable features and benefits as described in more detail below.

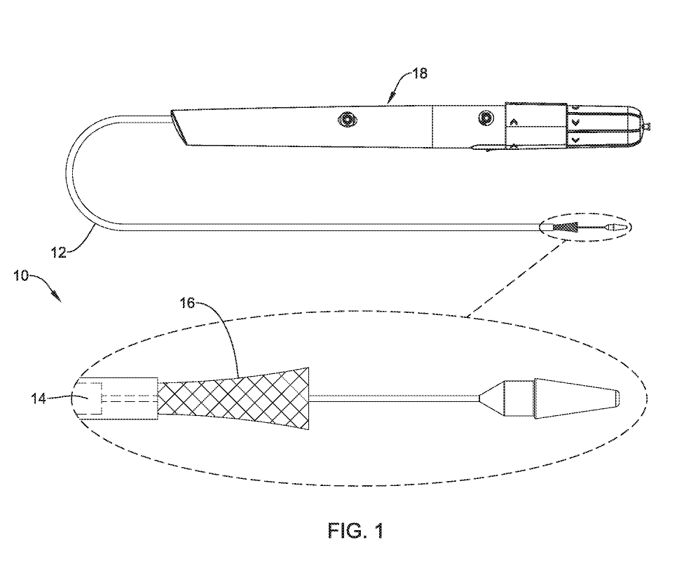

The figures illustrate selected components and/or arrangements of a medical device system 10, shown schematically in FIG. 1 for example. It should be noted that in any given figure, some features of the medical device system 10 may not be shown, or may be shown schematically, for simplicity. Additional details regarding some of the components of the medical device system 10 may be illustrated in other figures in greater detail. A medical device system 10 may be used to deliver and/or deploy a variety of medical devices to one or more locations within the anatomy. In at least some embodiments, the medical device system 10 may include a medical implant delivery system that can be used for percutaneous delivery of a medical implant 16, such as a replacement heart valve (e.g., a replacement aortic valve delivery system). This, however, is not intended to be limiting as the medical device system 10 may also be used for other interventions including valve repair, valvuloplasty, and the like, or other similar interventions.

The medical device system 10 may generally be described as a catheter system that includes an elongate outer sheath 12, an elongate inner sheath or catheter 14 (a portion of which is shown in FIG. 1 in phantom line) extending at least partially through a lumen of the outer sheath 12, and a medical implant 16 (e.g., a replacement heart valve implant, for example, which term may be used interchangeably with the term “medical implant” herein) which may be releasably coupled to the inner sheath or catheter 14 and disposed within a lumen of the outer sheath 12 during delivery of the medical implant 16.

In some embodiments, a handle 18 may be disposed at a proximal end of the outer sheath 12 and/or the inner sheath or catheter 14 and may include one or more actuation means associated therewith. The handle 18 may be configured to manipulate the position of the outer sheath 12 relative to the inner sheath or catheter 14 and/or aid in the deployment of the medical implant 16. In some embodiments, the medical device system 10 may include a nose cone disposed at a distal end of a guidewire extension tube, wherein the guidewire extension tube may extend distally from the inner sheath or catheter 14 and/or the outer sheath 12. In at least some embodiments, the nose cone may be designed to have an atraumatic shape and/or may include a ridge or ledge that is configured to abut a distal end of the outer sheath 12 during delivery of the medical implant 16.

In use, the medical device system 10 may be advanced percutaneously through the vasculature to an area of interest and/or a treatment location. For example, in some embodiments, the medical device system 10 may be advanced through the vasculature to a defective native valve (e.g., aortic valve, mitral valve, etc.). Alternative approaches to treat a defective aortic valve and/or other heart valve(s) are also contemplated with the medical device system 10. During delivery, the medical implant 16 may be generally disposed in an elongated and low profile “delivery” configuration within the lumen and/or a distal end of the outer sheath 12, as seen schematically in FIG. 1 for example. Once positioned, the outer sheath 12 may be retracted relative to the medical implant 16 and/or the inner sheath or catheter 14 to expose the medical implant 16. In some embodiments, the medical implant 16 may be disposed in an “everted” configuration while disposed within the lumen and/or the distal end of the outer sheath 12 and/or immediately upon exposure after retracting the outer sheath 12. The “everted” configuration may involve at least a portion of the valve leaflets (discussed below) of the medical implant 16 being disposed outside of the tubular anchor member (discussed below) of the medical implant 16 during delivery, thereby permitting a smaller radial profile of the medical implant 16 and the use of a smaller overall profile of the outer sheath 12 and/or the medical device system 10. In some embodiments, the “delivery” configuration and the “everted” configuration may be substantially similar and/or may be used interchangeably.

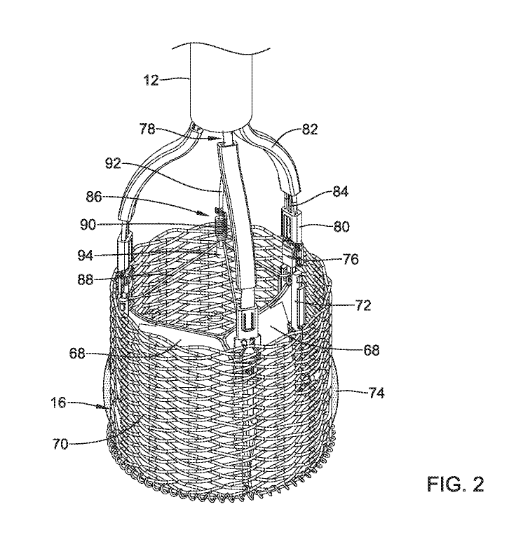

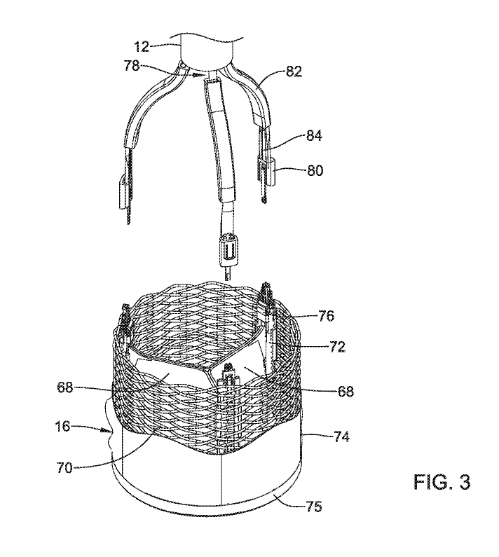

The medical implant 16 may be actuated using the handle 18 in order to translate the medical implant 16 into a generally shortened and radially-expanded larger profile “deployed” configuration, shown in FIG. 2 and described in more detail below, suitable for implantation within the anatomy at the area of interest and/or the treatment location. When the medical implant 16 is suitably deployed within the anatomy, the deployed medical implant 16 may be disconnected, detached, and/or released from the medical device system 10 and the medical device system 10 can be removed from the vasculature, leaving the deployed medical implant 16 in place in a “released” configuration, shown in FIG. 3, to function as, for example, a suitable replacement for the native valve. In at least some interventions, the medical implant 16 may be deployed and released within the native valve (e.g., the native valve is left in place and not excised). Alternatively, the native valve may be removed and the medical implant 16 may be deployed and released in its place as a replacement.

Turning back to FIG. 1, in some embodiments, the outer sheath 12 and/or the inner sheath or catheter 14 may take the form of an extruded polymer tube. Other forms are also contemplated including other polymer tubes, metallic tubes, reinforced tubes, or the like including other suitable materials such as those disclosed herein. In some embodiments, the outer sheath 12 and/or the inner sheath or catheter 14 may each be a singular monolithic or unitary member. In other embodiments, the outer sheath 12 and/or the inner sheath or catheter 14 may each include a plurality of portions or segments that are coupled together. In some embodiments, the outer sheath 12 and/or the inner sheath or catheter 14 may also be curved, for example adjacent to their respective distal ends. In some embodiments, the outer sheath 12 and/or the inner sheath or catheter 14 may have one or more sections with a differing hardness/stiffness (e.g., differing shore durometer).

In some embodiments, the inner sheath or catheter 14 may include one or more lumens extending longitudinally through the inner sheath or catheter 14. For example, the inner sheath or catheter 14 may include a first lumen, a second lumen, a third lumen, and a fourth lumen. Other configurations are also contemplated. In some examples, one or more of the lumens may extend along an entire length of and/or completely through the inner sheath or catheter 14. Other embodiments are contemplated, however, where one or more of the lumens extend along only a portion of the length of the inner sheath or catheter 14.

In some embodiments, disposed within the first lumen may be at least one actuator member 84, which may be used to reversibly actuate (e.g., translate axially or longitudinally and/or expand radially) the medical implant 16 between the “delivery” configuration and the “deployed” configuration, as explained in more detail herein. In some embodiments, the medical device system 10 may include at least one actuator member 84 extending from the handle 18 to the medical implant 16. In some embodiments, the at least one actuator member 84 may include a plurality of actuator members 84, two actuator members 84, three actuator members 84, four actuator members 84, or another suitable or desired number of actuator members 84. For the purpose of illustration only, the medical device system 10 and/or the medical implant 16 is shown with three actuator members 84 (e.g., FIGS. 2-3).

In some embodiments, disposed within the second lumen may be a pin release mandrel 92 and/or at least one release pin 88 (shown in FIG. 2 for example), although dedicated release pins 88 are not strictly necessary. The at least one release pin 88 may be secured to and/or integrally formed with the pin release mandrel 92. In some embodiments, the third lumen may be a guidewire lumen configured to slidably receive a guidewire therein. In some embodiments, the fourth lumen may be used to house a non-stretch wire or other reinforcing member. The exact form of the non-stretch wire or other reinforcing member may vary, and may serve to substantially prevent elongation of the inner sheath or catheter 14 during use. In some embodiments, the non-stretch wire or other reinforcing member may be embedded within the fourth lumen and/or the inner sheath or catheter 14. In addition, the non-stretch wire or other reinforcing member may extend to a position adjacent to the distal end but not fully to the distal end of the inner sheath or catheter 14. For example, in some embodiments, a short distal segment of the fourth lumen may be filled in with polymer material adjacent to the distal end of the inner sheath or catheter 14.

FIGS. 2 and 3 illustrate selected components of the medical device system 10 and/or the medical implant 16 in the “deployed” configuration (e.g., FIG. 2) and the “released” configuration (e.g., FIG. 3). The medical implant 16 includes a plurality of valve leaflets 68 (e.g., bovine pericardial, polymeric, etc.) which may be secured to a tubular anchor member 70 that is reversibly actuatable between the “delivery” configuration (e.g., FIG. 1) and the “deployed” configuration. In some embodiments, the tubular anchor member 70 may include a proximal end and a distal end. In at least some embodiments, the distal end of the tubular anchor member 70 may be interchangeably described as the “inflow” end or the “upstream” end of the tubular anchor member 70 and/or the medical implant 16. In at least some embodiments, the proximal end of the tubular anchor member 70 may be interchangeably described as the “outflow” end or the “downstream” end of the tubular anchor member 70 and/or the medical implant 16.

In some embodiments, the tubular anchor member 70 may be substantially cylindrical in shape or configuration. In some embodiments, the tubular anchor member 70 may define a central longitudinal axis extending from the proximal end of the tubular anchor member 70 to the distal end of the tubular anchor member 70, and/or a lumen extending through the tubular anchor member 70 along, parallel to, coaxial with, and/or coincident with the central longitudinal axis. In some embodiments, the tubular anchor member 70 may be and/or include a braid formed from one or more filaments or wires (e.g., a single filament or wire, two filaments or wires, etc.). Other shapes and/or configurations, including but not limited to a cut tube or stent, are also contemplated. Some suitable but non-limiting materials for the tubular anchor member 70, for example metallic materials or polymeric materials, are described below.

In some embodiments, the medical implant 16 may include a plurality of locking mechanisms attached to the tubular anchor member 70, the plurality of locking mechanisms being configured to secure the tubular anchor member 70 in the “deployed” configuration and/or the “released” configuration. In some embodiments, the at least one actuator member 84 may be configured to engage with the plurality of locking mechanisms and actuate the tubular anchor member 70 and/or the medical implant 16 between the “delivery” configuration, the “deployed” configuration, and/or the “released” configuration. In some embodiments, each and/or one actuator member 84 may correspond to, engage with, and/or actuate one locking mechanism. In some embodiments, each and/or one actuator member 84 may correspond to, engage with, and/or actuate more than one locking mechanism. Other configurations are also contemplated.

In some embodiments, the plurality of locking mechanisms may each comprise an axially movable post member 72, for example at the commissure portions of the valve leaflets 68 (the post member 72 may also be referred to as a “commissure post”, which may serve to secure the plurality of valve leaflets 68), and a buckle member 76 fixedly attached to the tubular anchor member 70 (e.g., along an interior surface of the tubular anchor member 70). In some embodiments, each of the plurality of valve leaflets 68 may be secured to the tubular anchor member 70 at and/or using at least one post member 72. In some embodiments, each of the plurality of valve leaflets 68 may be secured to two adjacent post members 72 at opposing sides of the valve leaflets 68. In at least some embodiments, the medical implant 16 may include a plurality of post members 72 and a corresponding plurality of buckle members 76. Other configurations and correspondences are also contemplated. In the illustrated example(s), the medical implant 16 includes three valve leaflets 68 secured to the tubular anchor member 70 with three post members 72 and three corresponding buckle members 76. The plurality of valve leaflets 68 may also be secured to the distal end and/or the inflow end of the tubular anchor member 70. The plurality of post members 72, in turn, may be secured to the tubular anchor member 70 (e.g., along an interior surface of the tubular anchor member 70) with sutures or other suitable means.

In some embodiments, the at least one actuator member 84 may be configured to engage with the plurality of locking mechanisms and actuate the tubular anchor member 70 and/or the medical implant 16 between the “delivery” configuration, the “deployed” configuration, and/or the “released” configuration. In some embodiments, each actuator member 84 may be generally round, oblong, ovoid, rectangular, polygonal (e.g., two-sided, three-sided, four-sided, five-sided, six-sided, etc.) in shape. Other shapes, both regular and irregular, are also contemplated. In some embodiments, each actuator member 84 may be formed from a single piece of wire, round stock, or other suitable material, as discussed herein. In some embodiments, each actuator member 84 may be formed by further processing the single piece of wire, round stock, or other suitable material, such as by machining, stamping, laser cutting, or other suitable techniques. Some suitable but non-limiting materials for the at least one actuator member 84, for example metallic materials or polymeric materials, are described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the actuator member”, “the locking mechanism”, “the lumen”, or other features may be equally referred to all instances and quantities beyond one of said feature. For simplicity and clarity purposes, not all elements of the disclosed invention are necessarily shown in each figure or discussed in detail below. In some illustrative examples, only one of the at least one actuator member 84, only one of the plurality of the post members 72, only one of the plurality of the buckle members 76, etc., are shown and discussed (and/or all of the medical implant 16 and/or the tubular anchor member 70 may not be shown to facilitate understanding of certain elements). However, it will be understood that the following discussion may apply equally to any and/or all of the components for which there are more than one within the medical implant 16 (e.g., the at least one actuator member 84, the plurality of locking mechanisms, etc.) and/or the medical device system 10, unless explicitly stated to the contrary. Additionally, not all instances of some elements or features may be shown in each figure for clarity.

In some embodiments, the post member 72 may engage the buckle member 76 in the “deployed” configuration, and consequently and/or subsequently, in the “released” configuration. In some embodiments, the post member 72 may be axially and/or longitudinally spaced apart from the buckle member 76 in the “delivery” configuration and/or the “everted” configuration. Some suitable but non-limiting materials for the post member 72 and/or the buckle member 76, for example metallic materials or polymeric materials, are described below.

In some embodiments, a distal end of the post member 72 may be secured and/or attached (e.g., fixedly attached, movably attached, removably attached, etc.) to a distal portion of the tubular anchor member 70, such as by a suture, a tether, adhesives, or other suitable element. In some embodiments, the post member 72 may be movable relative to the tubular anchor member 70 and/or the buckle member 76. In some embodiments, the post member 72 may be axially or longitudinally movable relative to the tubular anchor member 70 and/or the buckle member 76. In some embodiments, the buckle member 76 may be fixedly attached to the tubular anchor member 70. Other embodiments are contemplated where the buckle member 76 may be movably or removably attached to the tubular anchor member 70. In some embodiments, the post member 72 may be secured or attached (e.g., fixedly attached, movably attached, removably attached, etc.) to the distal end and/or the inflow end of the tubular anchor member 70. In some embodiments, the buckle member 76 may be fixed or attached to a proximal portion of the tubular anchor member 70. In some embodiments, the buckle member 76 may be fixed or attached at and/or to the proximal end and/or the outflow end of the tubular anchor member 70.

As discussed above, in some embodiments, the medical implant 16 may include one or more of the plurality of valve leaflets 68 secured to the tubular anchor member 70 at, adjacent to, and/or using (at least in part) the plurality of post members 72. In some embodiments, the plurality of valve leaflets 68 may also be secured to the distal end and/or the inflow end of the tubular anchor member 70. As such, when the post member 72 is pulled proximally to engage the buckle member 76, the plurality of valve leaflets 68 and the distal end and/or the inflow end of the tubular anchor member 70 may also be pulled proximally relative to the buckle member 76, thereby transitioning the tubular anchor member 70 and/or the medical implant 16 from the “delivery” configuration and/or the “everted” configuration toward the “deployed” configuration.

In some embodiments, the plurality of valve leaflets 68 may be coupled and/or secured (e.g., to the post member 72, to the tubular anchor member 70, and/or back to themselves) using one or more sutures, threads, wires, filaments, or other suitable elements. In some embodiments, the plurality of valve leaflets 68 may be coupled and/or secured (e.g., to the post member 72, to the tubular anchor member 70, and/or back to themselves) using an adhesive, a bonding agent, or other suitable securing means. In some embodiments, the plurality of valve leaflets 68 may be coupled and/or secured (e.g., to the post member 72, to the tubular anchor member 70, and/or back to themselves) using a fabric, a textile, or other thin flexible material. In some embodiments, the plurality of valve leaflets 68 may be coupled and/or secured (e.g., to the post member 72, to the tubular anchor member 70, and/or back to themselves) using various combinations of the above-described means.

In some embodiments, the tubular anchor member 70 may have a total of three buckle members 76 and three post members 72 attached and/or secured thereto. Similarly, one actuator member 84 may be operatively associated with each post member 72 and buckle member 76, for a total of three actuator members 84 in the illustrated example(s). Other embodiments are contemplated where fewer or more buckle members 76, post members 72, actuator members 84, etc. may be utilized.

In some embodiments, a seal member 74 may be circumferentially disposed on and/or about a distal portion and/or an inflow portion of the tubular anchor member 70, as seen in FIG. 2 (partially cutaway) and FIG. 3 for example, and as the term suggests, may help to seal an exterior of the medical implant 16 and/or the tubular anchor member 70 within and/or against the area of interest upon deployment (e.g., in the “deployed” configuration and/or the “released” configuration), thereby preventing leakage around the medical implant 16 and/or the tubular anchor member 70. In some embodiments, the seal member 74 may be disposed about and/or radially outward of an outside surface of the tubular anchor member 70. In some embodiments, the seal member 74 may be disposed around a perimeter and/or on or against the exterior or outer surface of the tubular anchor member 70. In some embodiments, the seal member 74 may be coupled and/or secured at the distal end and/or the inflow end of the tubular anchor member 70.

In some embodiments, the seal member 74 may include a plurality of layers of polymeric material. Some suitable polymeric materials may include, but are not necessarily limited to, polycarbonate, polyurethane, polyamide, polyether block amide, polyethylene, polyethylene terephthalate, polypropylene, polyvinylchloride, polytetrafluoroethylene, polysulfone, and copolymers, blends, mixtures or combinations thereof. Other configurations and/or other suitable materials are also contemplated.

In some embodiments, a distal end of the seal member 74 may include a reinforcing band 75 fixedly attached to the seal member 74 at and/or adjacent the distal end and/or the inflow end of the tubular anchor member 70, as best seen in FIG. 3. In some embodiments, the reinforcing band 75 may be integrally formed with, incorporated into, adhered to, and/or at least partially embedded within the seal member 74. In some embodiments, the reinforcing band 75 may be formed from a woven or nonwoven fabric strip, a textile, or other thin flexible material. The reinforcing band 75 may provide tear resistance in the vicinity of sutures, filaments, or other attachment elements associated with components or aspects of the medical implant 16. In some embodiments, the seal member 74 and/or the reinforcing band 75 may extend distally and/or longitudinally beyond the distal end and/or the inflow end of the tubular anchor member 70.

In some embodiments, attachment between the medical implant 16 and the inner sheath or catheter 14 (and/or the outer sheath 12) may be effected through the use of a coupler 78. The coupler 78 may generally include a cylindrical base (not shown) that may be disposed about, attached to, and/or extending from a distal end of the inner sheath or catheter 14 (and/or the outer sheath 12). Projecting distally from the base is a plurality of fingers (e.g., two fingers, three fingers, four fingers, etc.) that are each configured to engage with the medical implant 16 at one of the plurality of the buckle members 76 (for example, at a proximal end of the buckle members 76), with the at least one actuator member 84 extending therethrough and engaging the post members 72. A collar 80 may be disposed about each of the fingers of the coupler 78 to further assist in holding together the fingers and the buckle members 76 in the “delivery” configuration and the “deployed” configuration. A guide 82 may be disposed over each of the fingers proximal of the collar 80 and may serve to keep the fingers of the coupler 78 associated with the actuator members 84 extending adjacent to (and axially slidable relative to) the respective fingers of the coupler 78. Finally, in some embodiments, a pin release assembly 86, as shown in FIG. 2 for example, may be a linking structure that keeps the post members 72, the buckle members 76, and the actuator member(s) 84 associated with one another. The pin release assembly 86 may include the plurality of release pins 88 that may be joined together (e.g. via a coiled connection 90) and held to the pin release mandrel 92 (with a ferrule 94, for example). As mentioned above, the pin release assembly 86 may not be present in all embodiments of the medical implant 16, and in at least some embodiments, may utilize one or more of various “pinless” release and/or locking mechanisms. Other suitable configurations are also contemplated. Some suitable but non-limiting materials for the coupler 78, the fingers, the collars 80, the guides 82, and/or the pin release assembly 86, for example metallic materials or polymeric materials, are described below.

During delivery, the medical implant 16 may be releasably secured at the distal end of the inner sheath or catheter 14 by the fingers of the coupler 78 being coupled with a projecting proximal end of the buckle members 76 (and being held in place with the collar 80 disposed over the connection) and by the actuator members 84 and the post members 72 being operatively secured together. After the medical implant 16 is advanced within the anatomy to the area of interest or the treatment location, the outer sheath 12 may be withdrawn (e.g., moved proximally relative to the inner sheath or catheter 14 and/or the medical implant 16) to expose the medical implant 16. Then, the actuator members 84 can be used to translate and “lock” the tubular anchor member 70 and/or the medical implant 16 in the “deployed” configuration by proximally retracting the actuator members 84 relative to the buckle members 76, the outer sheath 12, and/or the inner sheath or catheter 14 to pull the post members 72 into engagement with the buckle members 76. Finally, in some embodiments, the release pins 88 can be removed, thereby uncoupling the actuator members 84 from the post members 72, which allows the tubular anchor member 70 and/or the medical implant 16 to be separated from the medical device system 10 and left in the anatomy at the area of interest or the treatment location in the “released” configuration. In some embodiments, the release pins 88 and/or the pin release assembly 86 may not be present, and other and/or alternative means of releasing the medical implant 16 may be utilized, such as a displacement-based or distance-based means of releasing the medical implant 16.

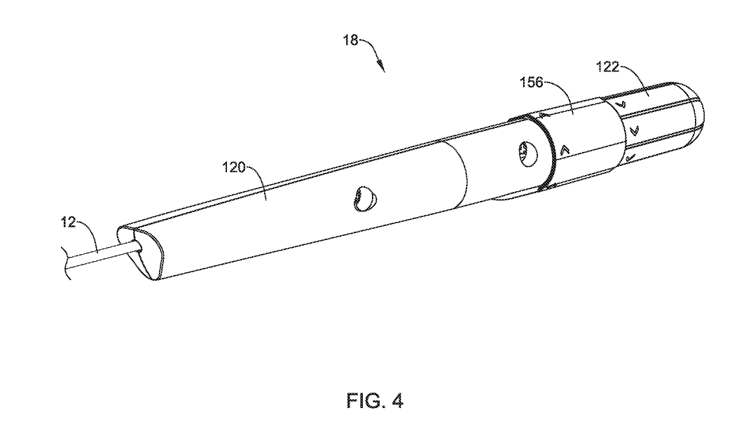

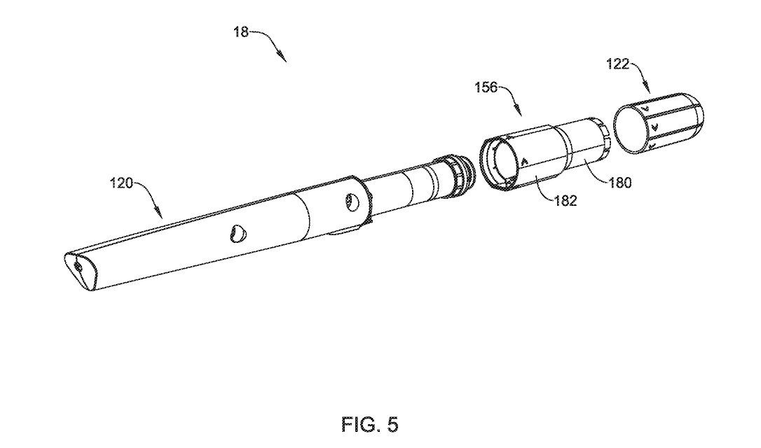

FIGS. 4-5 illustrate some aspects of an example handle 18. Here it can be seen that the handle 18 may include an elongated handle housing 120. The handle housing 120 may define a longitudinal axis extending from a proximal end of the handle housing 120 to a distal end of the handle housing 120. In some embodiments, the handle housing 120 may include a cavity disposed within the handle housing 120 (e.g., the handle housing 120 may be a hollow handle housing 120). A rotatable control knob 122 may be disposed about the handle housing 120 (e.g., at a proximal end of the handle housing 120) and may be used to actuate and/or move one or more of the components of the medical device system 10 (e.g., the outer sheath 12, the actuator members 84, etc.). In some embodiments, the rotatable control knob 122 may be rotatable about and/or with respect to the handle housing 120.

In some embodiments, a tubular collar member 156 may be disposed about a proximal portion of the handle housing 120. In some embodiments, the tubular collar member 156 may be rotatable about and/or with respect to the handle housing 120. In some embodiments, the rotatable control knob 122 may be disposed about a proximal portion 180 of the tubular collar member 156. In some embodiments, the tubular collar member 156 may be rotatable about and/or with respect to the rotatable control knob 122.

In some embodiments, the handle 18 may also include one or more apertures through the handle housing 120 and/or one or more flush ports accessible through the handle housing 120 that can be used to flush certain elements (e.g., components, lumens, etc.) of the medical device system 10.

In some embodiments, the handle 18 and/or the handle housing 120 may include a button mechanism actuatable between a first position and a second position relative to the tubular collar member 156, the handle housing 120, and/or the longitudinal axis of the handle housing 120 to release an interlock feature and permit the tubular collar member 156 to rotate about and/or relative to the elongated handle housing 120 to place the medical device system 10 in condition to translate and/or actuate the medical implant 16 from the “deployed” configuration to the “released” configuration. In some embodiments, the second position may be disposed radially inward of the first position. In some examples, in order to activate and/or actuate the button mechanism, the button mechanism must be moved, translated, and/or pressed radially inward toward the longitudinal axis of the handle housing 120 from the first position to the second position. In some embodiments, after activating and/or actuating the button mechanism to the second position, the tubular collar member 156 may be rotated about and/or relative to the elongated handle housing 120 to move one or more components of the medical device system 10 (e.g., the pin release mandrel 92, etc.).

In some embodiments, when the button mechanism is engaged with the tubular collar member 156 in the first orientation of the tubular collar member 156 (for example, with the button mechanism disposed in the first position), the tubular collar member 156 may be locked and/or prevented from rotating about and/or relative to the handle housing 120. In some embodiments, when the button mechanism is engaged with the tubular collar member 156 in the second orientation of the tubular collar member 156 (for example, with the button mechanism disposed in the first position), the tubular collar member 156 may be locked and/or prevented from rotating about and/or relative to the handle housing 120. In some embodiments, when the button mechanism is disposed is the second position, the tubular collar member 156 may be released and/or permitted to rotate about and/or with respect to the handle housing 120. Other means of locking and/or releasing the tubular collar member 156 relative to the handle housing 120 are also contemplated.

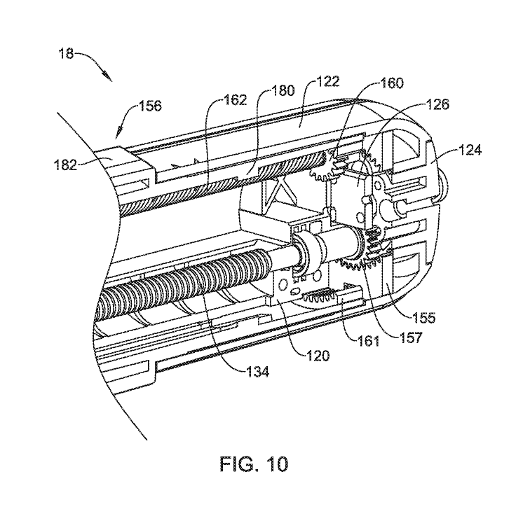

In some embodiments, the tubular collar member 156 may include a proximal portion 180 and a distal portion 182, as shown in FIG. 5 for example. In some embodiments, the proximal portion 180 may include a plurality of notches at a proximal end of the tubular collar member 156, the plurality of notches being configured to engage with a ring gear, as discussed below with respect to FIG. 10. In some embodiments, the rotatable control knob 122 may be disposed about and/or over the proximal portion 180 of the tubular collar member 156. In some embodiments, the distal portion 182 may be contoured and/or include indentations to facilitate grasping by a user to rotate the tubular collar member 156 about and/or relative to handle housing 120.

In some embodiments, the tubular collar member 156 may include one or more slots, ridges, and/or features disposed along an inner surface of the tubular collar member 156. The one or more slots, ridges, and/or features disposed along the inner surface of the tubular collar member 156 may serve to guide certain internal elements of the handle 18, prevent axial and/or rotational movement of certain elements of the handle 18 relative to the handle housing 120, and/or combinations thereof. In some embodiments, the one or more slots, ridges, and/or features disposed along the inner surface of the tubular collar member 156 may prevent premature movement and/or translation of certain elements of the handle 18 related to coordinated movement of multiple structures of the medical device system 10 described herein.

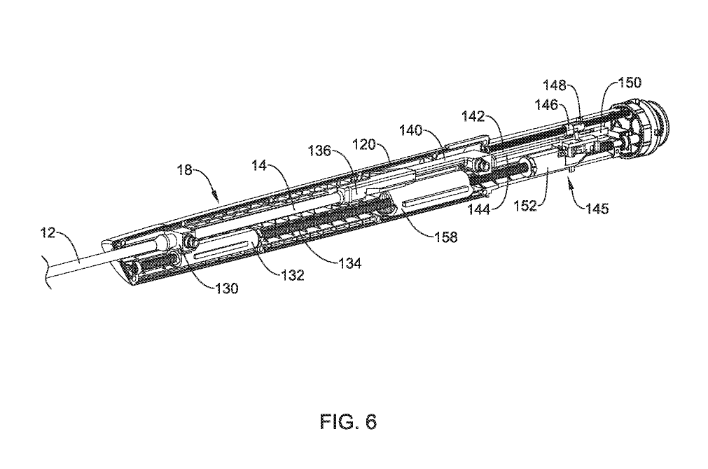

FIG. 6 illustrates the handle 18 with a portion of the handle housing 120 removed, exposing at least some of the components disposed within the cavity including at least some aspects of the deployment mechanism. Here it can be seen that a proximal end of the outer sheath 12 may be fixedly attached to a sheath adapter 130. The sheath adapter 130 may be fixedly attached to a sheath carriage 132, which may be threaded onto a first lead screw 134, wherein the first lead screw 134 extends through a lost motion barrel 158, which will be described in more detail below. A distal flush port may be disposed on the sheath adapter 130. In general, the distal flush port may provide access to the interior or lumen of the outer sheath 12 (e.g., access to space between the inner sheath or catheter 14 and the outer sheath 12) so that a clinician can flush fluid through the lumen of the outer sheath 12 to remove any unwanted materials (e.g., air, fluid, contaminants, etc.) therein prior to use of the medical device system 10. In at least some embodiments, the distal flush port may have a luer type connector (e.g., a one-way luer connector) that allows a device such as a syringe with a corresponding connector to be attached thereto for flushing.

In some embodiments, the inner sheath or catheter 14 may extend through and proximally from the sheath adapter 130. A proximal end of the inner sheath or catheter 14 may be attached (e.g., fixedly attached) to a diverter 136. The diverter 136 may be attached to a support body 140. In some embodiments, the diverter 136 and/or the support body 140 may have one or more passageways or lumens formed therein. In some embodiments, the at least one actuator member 84 and/or the pin release mandrel 92 (not visible in FIG. 6) may extend through respective (e.g., different) passageways or lumens formed in the diverter 136 and/or the support body 140. Alternatively, the proximal ends of the at least one actuator member 84 and/or the pin release mandrel 92 may each be fixedly attached to a shaft (e.g., solid in cross-section, tubular, etc.), and each of the shafts may extend through the one or more passageways or lumens. For example, a first shaft 142 and a second shaft 144 may extend through the passageways in the diverter 136 and/or the support body 140, and in some embodiments, the first shaft 142 extends through a first passageway and the second shaft 144 extends through a second passageway that is separate or distinct from the first passageway. In at least some embodiments, the first shaft 142 may be fixedly attached to the pin release mandrel 92 and/or the first shaft 142 may be integrally formed with the pin release mandrel 92. In at least some embodiments, the second shaft 144 may be fixedly attached to the at least one actuator member 84. It should be noted that at in least some embodiments of the medical device system 10, three actuator members 84 are utilized. In these embodiments, the three actuator members 84 may come together (e.g., may be brought into contact with one another or otherwise brought into relatively close proximity with one another) adjacent to the distal end of the inner sheath or catheter 14. At one or more positions along their length, the at least one actuator member 84 may be fixedly attached (e.g., welded, bonded, crimped, etc.) to one another.

In some embodiments, a hypotube may extend through the diverter 136 within a passageway therein and then be “diverted” around a portion of the diverter 136 and the support body 140, and ultimately be extended to a position at the proximal end of the handle 18 so as to provide a user access to a guidewire lumen (e.g., one of the lumens) of the inner sheath or catheter 14. A proximal flush port may be disposed on the support body 140 that can be used to flush the lumens of the inner sheath or catheter 14 and, for example, may function similarly to the distal flush port.

In some embodiments, the handle 18 may include a deployment mechanism configured to actuate the medical implant 16 positioned at the distal end of the medical device system 10. The deployment mechanism may be operatively engaged with the first lead screw 134. In some embodiments, the deployment mechanism may include a carriage assembly 145 movably disposed within the cavity of the handle 18 and/or the handle housing 120. In some embodiments, the carriage assembly 145 may be longitudinally movable between a distal position and a proximal position within the cavity by rotation of the rotatable control knob 122 with respect to the handle housing 120, as will be described in more detail below. In some embodiments, the carriage assembly 145 may include a carriage member 152, a first sliding member 146, a second sliding member 150, and a locking element 148 configured to releasably fix the first sliding member 146 and/or the second sliding member 150 to the carriage member 152. In some embodiments, the carriage assembly 145 and/or the carriage member 152 may be threaded onto and/or axially translatable along the first lead screw 134 disposed within the handle housing 120. In at least some embodiments, the carriage member 152, the second sliding member 150, the second shaft 144, and/or the at least one actuator member 84 may be collectively referred to as the deployment mechanism. The medical implant 16 may be releasably secured to the deployment mechanism by the at least one release pin 88, wherein proximal translation of the at least one release pin 88 releases the medical implant 16 from the medical device system 10.

At their respective proximal ends, the first shaft 142 and/or the pin release mandrel 92 may be secured (e.g., fixedly attached) to the first sliding member 146, and the second shaft 144 and/or the at least one actuator member 84 may be secured (e.g., fixedly attached) to the second sliding member 150. The connections between the various components may include a number of different types of connections including mechanical fixation (e.g., pinning, threading, interference fit, etc.), adhesive bonding, thermal bonding, etc. In some embodiments, the first sliding member 146 may be releasably fixed to and/or selectively slidable relative to the second sliding member 150 and/or the carriage member 152. In some embodiments, the first sliding member 146 may be releasably fixed and/or selectively locked to the second sliding member 150 and/or the carriage member 152 by the locking element 148, thereby preventing relative movement between the first sliding member 146 and the second sliding member 150. The second sliding member 150 may releasably fixed and/or selectively locked to the carriage member 152 by the locking element 148. Thus, rotation of the first lead screw 134 can cause axial movement and/or translation of the carriage assembly 145, the carriage member 152, the first sliding member 146, and/or the second sliding member 150 along the first lead screw 134 and/or relative to the handle housing 120. Thus, movement of the carriage assembly 145 from the distal position toward the proximal position may place the at least one actuator member 84 into tension, and/or the at least one actuator member 84 may also be axially translated relative to the handle housing 120 (via second shaft 144) by rotation of the first lead screw 134. Some additional details regarding this motion are discussed below.

In some embodiments, the handle 18 may include and/or define a stop feature (e.g., a hard stop, interference member, etc.) that prevents the carriage assembly 145 and/or the carriage member 152 from translating further in a proximal direction, and/or may provide tactile feedback (e.g., resistance to further rotation of the rotatable control knob 122) to the user indicating that the at least one actuator member 84 has been retracted proximally a sufficient distance to lock the post members 72 with the buckle members 76 (e.g., to actuate the medical implant 16 and/or the tubular anchor member 70 into the “deployed” configuration). To verify proper locking and/or positioning of the medical implant 16, a clinician may use an appropriate visualization technique (for example, to visualize the plurality of locking mechanisms, etc.).

The locking element 148 may be positioned adjacent to first sliding member 146 to selectively lock the first sliding member 146 to the second sliding member 150. In order to allow the first shaft 142 and/or the pin release mandrel 92 to be proximally retracted to pull the at least one release pin 88, the locking element 148 can be rotated or otherwise moved to a secondary position or configuration. When in this secondary position or configuration, the locking element 148 no longer forms a barrier to further movement of, for example, the first sliding member 146 and the first shaft 142 and/or the pin release mandrel 92 relative to the second sliding member 150. Accordingly, with the locking element 148 no longer acting as an impediment, the first sliding member 146 and the first shaft 142 and/or the pin release mandrel 92 can be proximally retracted to facilitate deployment of the medical implant 16 by allowing the at least one release pin 88 to be pulled.

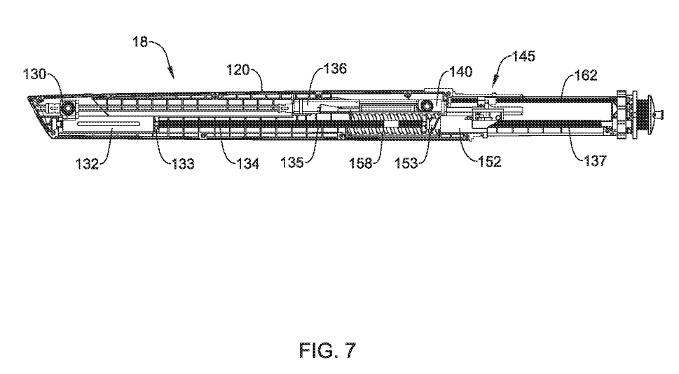

Turning to FIGS. 7-9, the handle 18 may generally be configured for coordinated movement of multiple structures of the medical device system 10. For example, the handle 18 may be configured to allow a user to move the outer sheath 12 (e.g., relative to the inner sheath or catheter 14), to move the second shaft 144 and/or the at least one actuator member 84, and/or to move the first shaft 142 and/or the pin release mandrel 92. Moreover, the handle 18 is configured so that the appropriate structure can be moved at the appropriate time during the intervention so that the medical implant 16 can be delivered in an efficient manner.

To help facilitate the coordinated movement, the handle 18 includes the lost motion barrel 158. The lost motion barrel 158 may be configured to engage the sheath carriage 132 and/or the carriage member 152 and/or screws associated with the sheath carriage 132 and/or the carriage member 152 at different times during the intervention to stop motion (e.g., create “lost motion” of the appropriate carriage). FIGS. 7-9 illustrate some of the coordinated motion achieved by the handle 18. It should be noted that some elements of the medical device system 10 are not shown in FIGS. 7-9 for clarity. For example, FIG. 7 illustrates a first state for the handle 18 wherein the outer sheath 12 (not shown) is extended distally relative to the inner sheath or catheter 14 (not shown), the medical implant 16 (not shown), and/or the handle housing 120 so as to fully sheath (e.g., contain) the medical implant 16 within the lumen and/or the distal end of the outer sheath 12. While in the first state, the sheath carriage 132 is positioned adjacent to the distal end of the handle 18. In addition, a rod screw 153 associated with the carriage member 152 is extended distally from the carriage member 152 and positioned within the lost motion barrel 158. Upon rotation of the rotatable control knob 122 (e.g., in a first or clockwise direction), the first lead screw 134 begins to rotate in the first or clockwise direction. Initial rotation of the first lead screw 134 causes the sheath carriage 132 to move along the first lead screw 134 in the proximal direction, resulting in proximal movement and/or translation of the outer sheath 12 relative to the inner sheath or catheter 14, the medical implant 16, and/or the handle housing 120 (e.g., “unsheathing” the medical implant 16). This initial rotation of the first lead screw 134 may also cause the rod screw 153 to rotate. This may be because, for example, a knob or projection on the rod screw 153 may be engaged with a helical thread disposed along an interior surface of the lost motion barrel 158. However, because the rod screw 153 is spaced from the carriage member 152, it does not exert a force onto the carriage member 152. Thus, initial rotation of the rotatable control knob 122 does not result in axial translation of the carriage member 152 and, instead, only results in axial translation of the sheath carriage 132 and rotation (and/or translation) of the rod screw 153. As such, initial rotation of the first lead screw 134 in the first or clockwise direction causes proximal translation of the outer sheath 12 relative to the inner sheath or catheter 14 without actuating the medical implant 16.

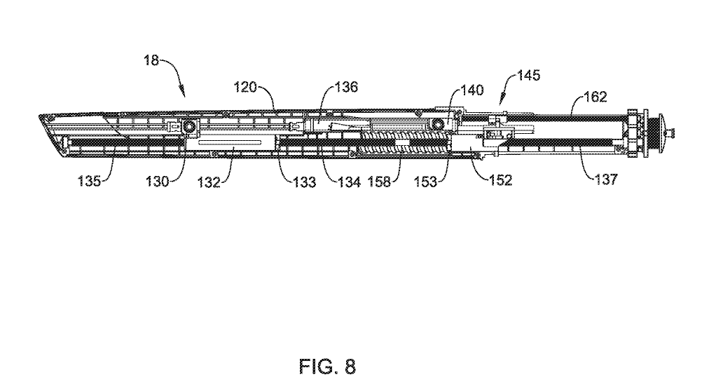

Eventually, the rod screw 153 (e.g., the knob formed therein) reaches an essentially linear thread or pathway formed at the proximal end of the lost motion barrel 158. The linear thread allows the rod screw 153 to axially translate proximally along the first lead screw 134 to a position where the rod screw 153 contacts (e.g., is threaded within and abuts) the carriage member 152, as shown in FIG. 8 for example. In doing so, the rod screw 153 can contact and axially translate the carriage member 152 proximally as the first lead screw 134 rotates in the first or clockwise direction. Accordingly, further rotation of the first lead screw 134 in the first or clockwise direction not only causes the sheath carriage 132 to move proximally but also causes the deployment mechanism and/or the carriage member 152 to move proximally.

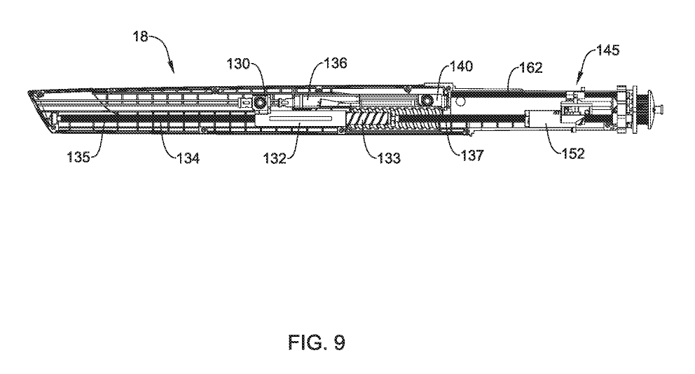

When the sheath carriage 132 reaches the lost motion barrel 158, a sheath carriage screw 133 of the sheath carriage 132 enters the lost motion barrel 158, as shown in FIG. 9 for example. This may occur in a manner similar to how the rod screw 153 threads and unthreads with the helical thread formed along the lost motion barrel 158. For example, while the sheath carriage 132 is translating axially, the sheath carriage screw 133 may follow an essentially linear thread or pathway formed along and/or adjacent to the lost motion barrel 158. Upon reaching the lost motion barrel 158, the sheath carriage screw 133 (e.g., a knob or projection formed thereon) may shift into engagement with the helical thread within the lost motion barrel 158 and rotate. This rotation “unthreads” the sheath carriage screw 133 from the sheath carriage 132. Accordingly, additional and/or subsequent rotation of the first lead screw 134 in the first or clockwise direction results in continued axial translation proximally of the deployment mechanism and/or the carriage member 152 while motion of the sheath carriage 132 ceases. As such, subsequent rotation of the first lead screw 134 in the first or clockwise direction causes the deployment mechanism to actuate the medical implant 16 without proximal translation of the outer sheath 12 relative to the inner sheath or catheter 14.

In at least some embodiments, the first lead screw 134 has a plurality of portions, for example a first portion 135 and a second portion 137, as seen in FIGS. 7-9, each with a differing pitch to its thread relative to one another. This may allow the sheath carriage 132 and/or the carriage member 152 to travel at different rates along first lead screw 134. For example, the pitch of first lead screw 134 along which the sheath carriage 132 translates may be generally more spaced or slanted than at positions adjacent to the carriage member 152. Accordingly, the coordinated movement of the sheath carriage 132 and/or the carriage member 152 also may be configured so that the sheath carriage 132 translates along the first lead screw 134 at a greater rate than the carriage member 152. Other configurations are contemplated where the above-mentioned configuration is reversed as well as further configurations where the pitch of first lead screw 134 is essentially constant or includes a number of different pitch regions.

Sufficient proximal retraction of the carriage member 152, for example as shown in FIG. 9, may result in the second shaft 144 and/or the at least one actuator member 84 being sufficiently retracted so that the post members 72 can engage and lock with the buckle members 76. When the clinician is satisfied that locking is complete (e.g., after verification via an appropriate visualization technique), the clinician may actuate the locking element 148 to the secondary position or configuration. The second lead screw 162 may extend through and/or be engaged with the first sliding member 146, wherein rotation of the second lead screw 162 produces proximal axial translation of the first sliding member 146 relative to the second sliding member 150 and/or the carriage member 152 with the locking element 148 to the secondary position or configuration. Proximal axial translation of the first sliding member 146 may produce corresponding proximal axial translation of the first shaft 142 and/or the pin release mandrel 92 to pull the at least one release pin 88 to disconnect and/or detach the at least one actuator member 84 from the post members 72 (and subsequently detaching the coupler 78 from the buckle members 76), thereby leaving the medical implant 16 in the “released” configuration. For example, proximal translation of the at least one release pin 88 may at least partially release the medical implant 16 from the medical device system 10.

Similar to the discussion above, rotation of the rotatable control knob 122 is a second direction (e.g., counterclockwise) opposite the first or clockwise direction causes rotation of the first lead screw 134 in the second or counterclockwise direction, for example, to translate and/or actuate the medical implant 16 and/or the tubular anchor member 70 toward and/or to the “delivery” configuration and/or to re-sheath the medical implant 16 within the lumen of the outer sheath 12. Translating and/or actuating the medical implant 16 and/or the tubular anchor member 70 toward and/or to the “delivery” configuration may be useful for repositioning the medical implant 16 at the area of interest if the initial positioning was improper or inadequate.

FIG. 10 illustrates a detailed partial-sectional view of selected elements of the handle 18. The rotatable control knob 122 is disposed over the proximal portion 180 of the tubular collar member 156. A distal end of the rotatable control knob 122 may face and/or contact a proximal face of the distal portion 182 of the tubular collar member 156. The rotatable control knob 122 may include an end cap 124 configured to secure the rotatable control knob 122 to the handle housing 120. For example, the end cap 124 may be attached to an internal portion of the handle housing 120 and/or to a securement plate 126 removably disposed within the handle housing 120. The end cap 124 may include at least one lumen extending through the end cap 124 for passage of a guidewire into the hypotube extending through the diverter 136 and around a portion of the diverter 136 and the support body 140 and/or into the guidewire lumen.

As may be seen in FIG. 10 for example, the first lead screw 134 and/or the second lead screw 162 may be spaced apart from the rotatable control knob 122 and/or the tubular collar member 156 within the handle housing 120. The first lead screw 134 and/or the second lead screw 162 are rotatable independently of and/or relative to the handle housing 120. In at least some embodiments, one full rotation of the rotatable control knob 122 produces more than one full rotation of the first lead screw 134. For example, rotation of the rotatable control knob 122 at a first rate (e.g., revolutions per minute, etc.) may produce rotation of the first lead screw 134 at a second rate higher than the first rate. In at least some embodiments, one full rotation of the tubular collar member 156 produces more than one full rotation of the second lead screw 162. For example, rotation of the tubular collar member 156 at a third rate (e.g., revolutions per minute, etc.) may produce rotation of the second lead screw 162 at a fourth rate higher than the third rate.

The rotatable control knob 122 may include a ring gear 155 and/or a plurality of internal teeth disposed along an inner surface of the rotatable control knob 122 proximate a proximal portion of the rotatable control knob 122. In some embodiments, the ring gear 155 and/or the plurality of internal teeth may be fixedly attached to the rotatable control knob 122. In some embodiments, the ring gear 155 and/or the plurality of internal teeth may be integrally formed with the rotatable control knob 122. Similarly, the tubular collar member 156 may include a second ring gear 161 and/or a second plurality of internal teeth disposed along an inner surface of the tubular collar member 156 proximate a proximal end of the tubular collar member 156. In some embodiments, the plurality of notches at the proximal end of the proximal portion 180 of the tubular collar member 156 may engage with the ring gear 155. In some embodiments, the second ring gear 161 and/or the second plurality of internal teeth may be fixedly attached to the tubular collar member 156. In some embodiments, the second ring gear 161 and/or the second plurality of internal teeth may be integrally formed with the tubular collar member 156.

The first lead screw 134 may include a pinion gear 157 and/or a plurality of external teeth at a proximal end of the first lead screw 134. In some embodiments, the pinion gear 157 and/or the plurality of external teeth may be fixedly attached to the first lead screw 134. In some embodiments, the pinion gear 157 and/or the plurality of external teeth may be integrally formed with the first lead screw 134. Similarly, the second lead screw 162 may include a second pinion gear 160 and/or a second plurality of external teeth at a proximal end of the second lead screw 162. In some embodiments, the second pinion gear 160 and/or the second plurality of external teeth may be fixedly attached to the second lead screw 162. In some embodiments, the second pinion gear 160 and/or the second plurality of external teeth may be integrally formed with the second lead screw 162.

The pinion gear 157 and/or the plurality of external teeth may engage with the ring gear 155 and/or the plurality of internal teeth to operatively connect the first lead screw 134 to the rotatable control knob 122. As such, rotation of the rotatable control knob 122 and the ring gear 155 and/or the plurality of internal teeth associated therewith may produce rotation of the first lead screw 134 and the pinion gear 157 and/or the plurality of external teeth associated therewith. In at least some embodiments, the securement plate 126 and/or the end cap 124 may maintain the pinion gear 157 and/or the plurality of external teeth in position within the handle housing 120 and/or aligned with the ring gear 155 and/or the plurality of internal teeth. Similarly, the second pinion gear 160 and/or the second plurality of external teeth may engage with the second ring gear 161 and/or the second plurality of internal teeth to operatively connect the second lead screw 162 to the tubular collar member 156. As such, rotation of the tubular collar member 156 and the second ring gear 161 and/or the second plurality of internal teeth associated therewith may produce rotation of the second lead screw 162 and the second pinion gear 160 and/or the second plurality of external teeth associated therewith.

In at least some embodiments, the pinion gear 157 and/or the plurality of external teeth may be directly engaged with the ring gear 155 and/or the plurality of internal teeth. For example, the handle 18 may lack any intervening gears, gearing, teeth, or other structure(s) between the pinion gear 157 and/or the plurality of external teeth and the ring gear 155 and/or the plurality of internal teeth. The relative size of the pinion gear 157 and the ring gear 155 may define a relative rate of rotation (e.g., gear ratio) between the pinion gear 157 and the ring gear 155. Similarly, the second pinion gear 160 and/or the second plurality of external teeth may be directly engaged with the second ring gear 161 and/or the second plurality of internal teeth. For example, the handle 18 may lack any intervening gears, gearing, teeth, or other structure(s) between the second pinion gear 160 and/or the second plurality of external teeth and the second ring gear 161 and/or the second plurality of internal teeth. The relative size of the second pinion gear 160 and the second ring gear 161 may define a relative rate of rotation (e.g., gear ratio) between the second pinion gear 160 and the second ring gear 161.

In a brief summary of the operation of the medical device system 10, to initiate release of the medical implant 16 after actuating and/or translating the medical implant 16 and/or the tubular anchor member 70 into the “deployed” configuration using the rotatable control knob 122, the button mechanism may be depressed, actuated, and/or translated radially inward relative to the longitudinal axis of the handle housing 120 from the first position to the second position while the tubular collar member 156 is in the first orientation, thereby disengaging the button mechanism from a first aperture. Depressing, actuating, and/or translating the button mechanism from the first position to the second position may unlock the tubular collar member 156, thereby permitting relative movement (e.g., rotational movement, etc.) of the tubular collar member 156 with respect to the handle housing 120.

Next, the tubular collar member 156 may be rotated about and/or relative to the handle housing 120 from the first orientation to the second orientation. In some embodiments, when the tubular collar member 156 is in the first orientation, the locking element 148 may physically engage the second sliding member 150 and/or the carriage member 152, thereby locking the first sliding member 146, the second sliding member 150, and the carriage member 152 with respect to longitudinal and/or axial movement therebetween when the locking element 148 is in a locked orientation. When the locking element 148 is in the locked orientation, the first sliding member 146, which may be positioned within a slot or groove within the second sliding member 150, may be secured and/or disposed between the second sliding member 150 and the locking element 148 in a first position at and/or adjacent a distal end of the slot or groove within the second sliding member 150, thereby preventing axial and/or sliding movement of the first sliding member 146 relative to the second sliding member 150, the carriage member 152, and/or the handle housing 120.

Initial rotation of the tubular collar member 156 about the longitudinal axis and/or away from the first orientation may cause a protrusion extending radially inwardly from the inner surface of the tubular collar member 156 to contact and rotate the locking element 148 with respect to the second lead screw 162, the first sliding member 146, the second sliding member 150, and/or the handle housing 120, etc., out of engagement with the second sliding member 150 and/or the carriage member 152 to an unlocked orientation (e.g., the secondary position or configuration). After rotating the locking element 148 out of engagement with the second sliding member 150 and/or the carriage member 152 and/or to the unlocked orientation, the first sliding member 146 may be longitudinally and/or axially movable proximally relative to the second sliding member 150 and/or the carriage member 152 from the first position toward a second position at and/or adjacent a proximal end of the slot or groove in the second sliding member 150. In some embodiments, after releasing the first sliding member 146 and the second sliding member 150 from the carriage member 152, tension on the second shaft 144 and/or the at least one actuator member 84 may be released until the first sliding member 146 re-engages the second sliding member 150 at a proximal end of a slot formed in the second sliding member 150.

Further rotation of the tubular collar member 156 about and/or relative to the handle housing 120 in the first or clockwise direction may cause the second lead screw 162 to turn in the first or clockwise direction, thereby translating and/or axially moving the first sliding member 146 proximally within the slot or groove in the second sliding member 150 (and/or relative to the second sliding member 150) from the first position to the second position. In at least some embodiments, when the first sliding member 146 is in the second position, the first sliding member 146 abuts a portion of the second sliding member 150. In some embodiments, when the first sliding member 146 is in the second position, the first sliding member 146 abuts a distally-facing surface within the slot or groove of the second sliding member 150. Proximal translation and/or movement of the first sliding member 146 from the first position to the second position may axially retract and/or pull the first shaft 142 and/or the pin release mandrel 92 and/or the at least one release pin 88 to disconnect and/or disengage the at least one release pin 88 from the medical implant 16, the locking mechanism(s), and/or the post member(s) 72, thereby irreversibly detaching the at least one actuator member 84 from the medical implant 16.

In some embodiments, after the first sliding member 146 is in the second position within the slot or groove in the second sliding member 150, further and/or partial rotation of the tubular collar member 156 about and/or relative to the handle housing 120 in the first or clockwise direction may move and/or axially translate the second sliding member 150 proximally relative to the carriage member 152 and/or the handle housing 120. In some embodiments, after the first sliding member 146 is in the second position within the slot or groove in the second sliding member 150, further and/or partial rotation of the tubular collar member 156 about and/or relative to the handle housing 120 in the first or clockwise direction may move and/or axially translate the first sliding member 146 and the second sliding member 150 proximally together and/or simultaneously relative to the carriage member 152 and/or the handle housing 120. Axial translation of the second sliding member 150 proximally relative to the carriage member 152 may proximally retract the at least one actuator member 84 from the medical implant 16, the locking mechanism(s), and/or the post member(s) 72.

Upon achieving the second orientation of the tubular collar member 156, the button mechanism may extend, actuate, and/or translate from the second position radially outward to the first position within a second aperture, and may re-engage and/or lock the tubular collar member 156 with respect to the handle housing 120. Re-engagement of the button mechanism with the tubular collar member 156 in the second orientation may indicate to a user that the release process has been completed and/or that the medical implant 16 has been released from the medical device system 10.

The materials that can be used for the various components of the medical device system 10, the outer sheath 12, the inner sheath or catheter 14, the medical implant 16, the handle 18, etc. (and/or other systems disclosed herein) and the various elements thereof disclosed herein may include those commonly associated with medical devices. For simplicity purposes, the following discussion makes reference to the medical device system 10, the outer sheath 12, the inner sheath or catheter 14, the medical implant 16, the handle 18, etc. However, this is not intended to limit the devices and methods described herein, as the discussion may be applied to other elements, members, components, or devices disclosed herein, such as, but not limited to, the valve leaflets 68, the tubular anchor member 70, the post members 72, the seal member 74, the buckle members 76, the collars 80, the guides 82, the actuator members 84, the pin release assembly 86, the handle housing 120, the control knob 122, the end cap 124, the securement plate 126, the tubular collar member 156, the sheath adapter 130, the sheath carriage 132, the first lead screw 134, the diverter 136, the support body 140, the first shaft 142, the second shaft 144, the carriage assembly 145, the ring gear 155, the pinion gear 157, the lost motion barrel 158, the second pinion gear 160, the second ring gear 161, the second lead screw 162, etc., and/or elements or components thereof.

In some embodiments, the medical device system 10, the outer sheath 12, the inner sheath or catheter 14, the medical implant 16, the handle 18, etc., and/or components thereof (such as, but not limited to, the valve leaflets 68, the tubular anchor member 70, the post members 72, the seal member 74, the buckle members 76, the collars 80, the guides 82, the actuator members 84, the pin release assembly 86, the handle housing 120, the control knob 122, the end cap 124, the securement plate 126, the tubular collar member 156, the sheath adapter 130, the sheath carriage 132, the first lead screw 134, the diverter 136, the support body 140, the first shaft 142, the second shaft 144, the carriage assembly 145, the ring gear 155, the pinion gear 157, the lost motion barrel 158, the second pinion gear 160, the second ring gear 161, the second lead screw 162, etc.), may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical device system 10, the outer sheath 12, the inner sheath or catheter 14, the medical implant 16, the handle 18, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device system 10, the outer sheath 12, the inner sheath or catheter 14, the medical implant 16, the handle 18, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10, the outer sheath 12, the inner sheath or catheter 14, the medical implant 16, the handle 18, etc. to achieve the same result.

In some embodiments, a degree of Magnetic Resonance Imaging (MM) compatibility is imparted into the medical device system 10, the outer sheath 12, the inner sheath or catheter 14, the medical implant 16, the handle 18, etc. For example, the medical device system 10, the outer sheath 12, the inner sheath or catheter 14, the medical implant 16, the handle 18, etc., and/or components or portions thereof, may be made of a material that does not substantially distort the image and create substantial artifacts (e.g., gaps in the image). Certain ferromagnetic materials, for example, may not be suitable because they may create artifacts in an Mill image. The medical device system 10, the outer sheath 12, the inner sheath or catheter 14, the medical implant 16, the handle 18, etc., or portions thereof, may also be made from a material that the MM machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device system 10, the outer sheath 12, the inner sheath or catheter 14, the medical implant 16, the handle 18, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device system 10, the outer sheath 12, the inner sheath or catheter 14, the medical implant 16, the handle 18, etc. and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the medical device system 10, the outer sheath 12, the inner sheath or catheter 14, the medical implant 16, the handle 18, etc. may include and/or be formed from a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyl s, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the medical device system 10, the outer sheath 12, the inner sheath or catheter 14, the medical implant 16, the handle 18, etc. may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made to details, particularly in matters of shape, size, and arrangement of steps, without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A handle for a medical device system, comprising:
a hollow elongate handle housing;
a first lead screw disposed within the hollow elongate handle housing;
a deployment mechanism configured to actuate a medical implant positioned at a distal end of the medical device system, the deployment mechanism being operatively engaged with the first lead screw; and
a control knob rotatably disposed around a proximal end of the hollow elongate handle housing;
wherein the first lead screw is spaced apart from the control knob;
wherein rotation of the control knob in a first direction causes rotation of the first lead screw in the first direction.

2. The handle of claim 1, wherein one full rotation of the control knob produces more than one full rotation of the first lead screw.

3. The handle of claim 1, wherein the control knob includes a ring gear disposed along an inner surface of the control knob.

4. The handle of claim 3, wherein the ring gear is fixedly attached to the control knob.

5. The handle of claim 3, wherein the first lead screw includes a pinion gear at a proximal end of the first lead screw.

6. The handle of claim 5, wherein the pinion gear is directly engaged with the ring gear.

7. The handle of claim 5, wherein the pinion gear is fixedly attached to the proximal end of the first lead screw.

8. The handle of claim 1, wherein rotation of the control knob in a second direction opposite the first direction causes rotation of the first lead screw in the second direction.

9. The handle of claim 1, further comprising a tubular collar disposed about a proximal portion of the hollow elongate handle housing, the tubular collar being rotatable relative to the hollow elongate handle housing and the control knob.

10. A medical device system, comprising:
an elongate outer sheath;
an elongate inner sheath disposed within a lumen of the outer sheath; and
a handle disposed at a proximal end of the outer sheath, comprising:
a hollow elongate handle housing;
a first lead screw disposed within the hollow elongate handle housing;
a deployment mechanism configured to actuate a medical implant positioned at a distal end of the inner sheath, the deployment mechanism being operatively engaged with the first lead screw; and
a control knob rotatably disposed around a proximal end of the hollow elongate handle housing;
wherein the first lead screw is spaced apart from the control knob;
wherein rotation of the control knob in a first direction causes rotation of the first lead screw in the first direction;
wherein initial rotation of the first lead screw in the first direction causes proximal translation of the outer sheath relative to the inner sheath without actuating the medical implant.

11. The medical device system of claim 10, wherein subsequent rotation of the first lead screw in the first direction causes the deployment mechanism to actuate the medical implant without proximal translation of the outer sheath relative to the inner sheath.

12. The medical device system of claim 10, wherein the medical implant is a replacement heart valve implant.

13. The medical device system of claim 27, wherein actuation of the replacement heart valve implant shifts the replacement heart valve implant from an elongated delivery configuration to an expanded deployed configuration.

14. The medical device system of claim 10, wherein the medical implant is releasably secured to the deployment mechanism by at least one release pin.

15. The medical device system of claim 14, wherein proximal translation of the at least one release pin releases the medical implant from the medical device system.

16. A handle for a medical device system, comprising:
a hollow elongate handle housing;
a first lead screw disposed within the hollow elongate handle housing, the first lead screw including a plurality of external teeth disposed at a proximal end of the first lead screw;
a deployment mechanism configured to actuate a medical implant positioned at a distal end of the medical device system, the deployment mechanism being operatively engaged with the first lead screw; and
a control knob rotatably disposed around a proximal end of the hollow elongate handle housing, the control knob including a plurality of internal teeth proximate a proximal portion of the control knob;
wherein the plurality of external teeth is directly engaged with the plurality of internal teeth;
wherein rotation of the control knob in a first direction causes rotation of the first lead screw in the first direction.

17. The handle of claim 16, wherein the plurality of external teeth is integrally formed with the first lead screw.

18. The handle of claim 16, wherein the plurality of internal teeth is integrally formed with the control knob.

19. The handle of claim 16, wherein the first drive screw is rotatable independently of the hollow elongate handle housing.

20. The handle of claim 16, wherein rotation of the control knob at a first rate produces rotation of the first lead screw at a second rate higher than the first rate.
